(19) United States
(12) Patent Application Publication
(10) Pub. No.: US 2007/0219516 A1
(43) Pub. Date: Sep. 20, 2007

(54) X-ray detectable element for association with surgical absorbent substrates and method of making

(75) Inventors: Harish A. Patel, Norfolk, MA (US); David Fink, Franklin, MA (US)

Correspondence Address: CARTER, DELUCA, FARRELL & SCHMIDT, LLP, 445 BROAD HOLLOW ROAD, SUITE 225, MELVILLE, NY, 11704, US

(73) Assignee: Tyco Healthcare Group LP

(21) Appl. No.: 11/714,310

(22) Filed: Mar. 6, 2007

Related U.S. Application Data
(60) Provisional application No. 60/782,141, filed on Mar. 14, 2006.

(51) Int. Cl. A61F 13/15

(52) U.S. Cl. 604/362

(57) ABSTRACT

An x-ray detectable element for use in association with a surgical absorbent substrate includes a detectable element having an extruded sheath at least partially encasing an elongated radiopaque core. The absorbent substrate may include, woven and non-woven, surgical sponges and gauze.

### CROSS-REFERENCE TO RELATED APPLICATIONS

This patent application claims priority to and the benefit of U.S. Provisional Patent Application No. 60/782,141, filed in the U.S. Patent and Trademark Office on Mar. 14, 2006.

### TECHNICAL FIELD

The present disclosure relates to an x-ray detectable element, and more particularly, relates to an x-ray detectable element for use in association with surgical absorbent substrates.

### BACKGROUND

Radiopaque elements are associated with surgical absorbent substrates to assist in their detection, retrieval and accounting during surgical procedures. As appreciated, any foreign matter mistakenly left within a surgery site may cause serious complications during the patient's recovery. For example, a forgotten sponge left at the surgical site may lead to serious infection or death. Subsequent removal of a forgotten sponge requires the reopening of the surgery site. This reopening could lead to infection, further scarring, or other more serious complications. It is therefore necessary to provide an absorbent substrate that is capable of being detected prior to the closing of the surgery site.

### SUMMARY

Accordingly, the present disclosure relates to an x-ray detectable element for use in association with a surgical absorbent substrate. The detectable element has an extruded sheath at least partially encasing an elongated radiopaque core. The absorbent substrate may include, woven and non-woven, surgical sponges and gauze.

The radiopaque core of the x-ray detectable element is an elongated filament or thread containing anywhere from 30% to 90% opacifying agent or agents. The opacifying agent or agents may be combined, or blended, with one or more elastomeric materials, and/or polymers, to form the core. Such an elastomeric polymer is polyvinylchloride (PVC). A common opacifying agent is barium sulfate, BaSO₄. The cross-section of the radiopaque core is generally circular permitting for uniform visibility of the element under x-ray throughout the length of the element. Alternatively, the cross-section of the radiopaque core may be any shape, including square, rectangular and oval. The cross-section of the radiopaque core may also be lobed or multilobed. The radiopaque core may be of a continuous length within the sheath, or the radiopaque core may be intermittently spaced in the longitudinal direction for easier recognition over other structures in the surgical field, such a pacemaker wires.

The sheath of the x-ray detectable element is extruded about the radiopaque core, or both the radiopaque core and the sheath are extruded together, e.g., co-extruded. The sheath at least partial encases the radiopaque core. The sheath may be colored for improved visibility within a blood red environment. The sheath may also be composed of hydrophobic material so as to repel blood or other fluids encountered in the surgical field. The sheath may be anywhere from 80% to 99% elastomeric material.

The sheath may further be coated or impregnated with leachable microbial agents. Yet another variation of the element may have a sheath impregnated or coated with magnetic material for detection by an electromagnetic device. Additional opacifying agent or agents may be added to the sheath for improved detectability.

The sheath may be radially partitioned into sheath regions in the longitudinal direction for improved visibility. One or more of the sheath regions may be of different colors and/or compositions. Sheath regions of different colors permit improved visibility of the element, while embedded opacifying agent or agents and magnetic material increase the detectability of the element. An additional layer at least partially encasing the sheath may also be added to the element for increased visibility. The additional layer may be composed of any combination of any or all of the above mentioned materials or colors.

### BRIEF DESCRIPTION OF THE DRAWINGS

Preferred embodiments of the present disclosure will be better appreciated by reference to the drawings wherein:

FIG. 1A is an axial cross-sectional view of an x-ray detectable element in accordance with the principles of the present disclosure;

FIG. 1B is a longitudinal cross-sectional view of the detectable element of FIG. 1A;

FIG. 2A is an axial cross-sectional view of an alternate embodiment of the x-ray detectable element of FIGS. 1A and 1B;

FIG. 2B is a longitudinal cross-sectional view of the detectable element of FIG. 2A;

FIG. 3A is an axial cross-sectional view of an alternate embodiment of the x-ray detectable element of FIGS. 1A and 1B;

FIG. 3B and 3C are longitudinal cross-sectional views of the x-ray detectable element of FIG. 3A taken along the lines b-b and c-c of FIG. 3A, respectively;

FIG. 4A is an axial cross-sectional view of an alternate embodiment of the x-ray detectable element of FIGS. 1A and 1B;

FIG. 4B is a side view of the x-ray detectable element of FIG. 4A;

FIG. 5 is an axial cross-sectional view of an alternate embodiment of the x-ray detectable element of FIGS. 1A and 1B;

FIG. 6 is a longitudinal cross-sectional view of an alternate embodiment of the x-ray detectable element of FIGS. 1A and 1B;

FIG. 7 is a longitudinal cross-sectional view of an alternate embodiment of the x-ray detectable element of FIGS. 1A and 1B;

FIG. 8 is a longitudinal cross-sectional view of an alternate embodiment of the x-ray detectable element of FIGS. 1A and 1B;

FIG. 9 is a fragmentary plan view of a surgical absorbent substrate in association with an x-ray detectable element constructed in accordance with the present disclosure;

FIG. 10 is a fragmentary plan view of a surgical absorbent substrate in which an x-ray detectable element is formed as an integral part of the substrate; and

FIGS. 11A and 11B are plan views of surgical absorbent substrates having differing patterns formed by x-ray detectable elements.

### DESCRIPTION OF THE PREFERRED EMBODIMENTS

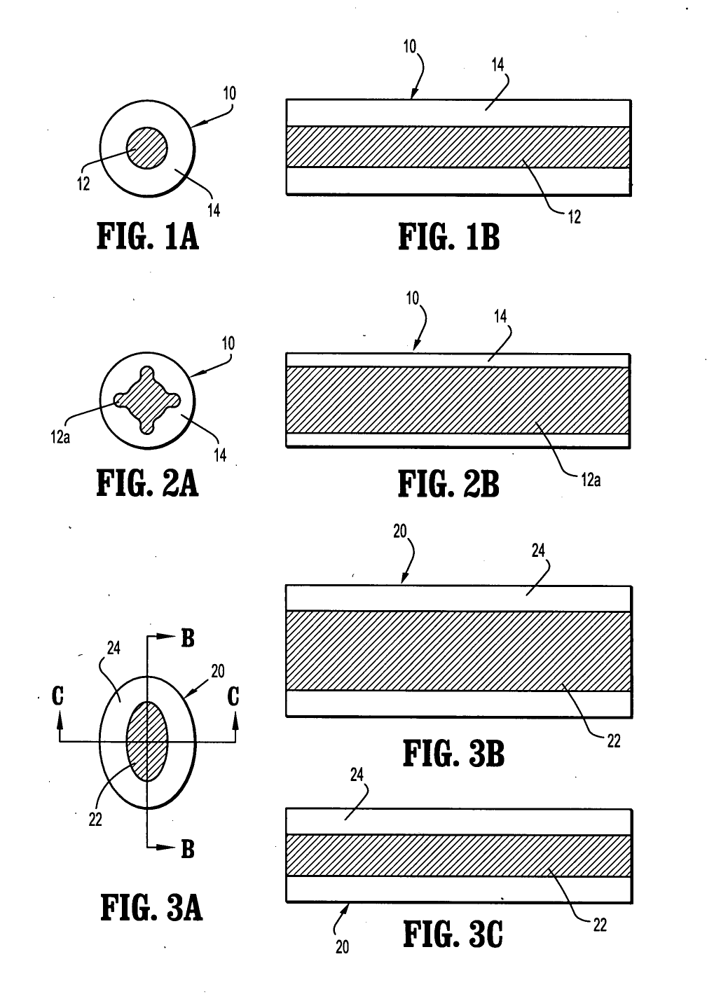

Referring now to the drawings wherein like reference numerals illustrate similar components throughout the several views, FIGS. 1A and 1B illustrate one preferred embodiment of the present disclosure. X-ray detectable element 10 is contemplated for use in association with a surgical absorbent substrate, such as a sponge, gauze, or the like. X-ray detectable element 10 includes radiopaque core 12 partially encased within sheath 14. Radiopaque core 12 is an elongated filament or thread containing, preferably, anywhere from 30% to 90% opacifying agent or agents. Other percentages for the opacifying agents are also contemplated. The opacifying agent or agents may be combined, or blended, with one or more other elastomeric materials or polymers to form radiopaque core 12. Such an elastomeric polymer is polyvinylchloride (PVC). A common opacifying agent is barium sulfate, BaSO₄. In one preferred embodiment, radiopaque core 12 has a circular cross-section which provides uniform visibility of the element along its length when viewed under x-ray. Radiopaque core 12 may, however, take any form, including square, rectangular, oval, lobed and multi-lobed 12a (FIGS. 2A and 2B).

Sheath 14 of x-ray detectable element 10 at least partially encases radiopaque core 12 and, preferably, fully encloses radiopaque core 12. Sheath 14 may be fabricated from an elastomeric material, polymeric material, or the like. Sheath 14 may be colored for improved visibility. For example, the colors of sheath 14 are chosen to contrast element 10 with linens and dressings found in the surgical field. Such colors are inclusive of blue or green, although other colors are contemplated. Sheath 14 may also be white to contrast element 10 with the blood present in the surgical site. Sheath 14 may also be composed of hydrophobic material so as to repel blood or other fluids encountered in the surgical field. Sheath 14 may comprise anywhere from 80% to 99% elastomeric material although other compositions are also envisioned. The percentage of elastomeric material used in sheath 14 alters the strength, elasticity, heat bondability to the absorbent substrate, and integrity of element 10.

Sheath 14 may further be coated, or impregnated, with leachable microbial agents. Sheath 14 may also be impregnated, or coated, with magnetic material for detection by an electro-magnetic device. Additional opacifying agent or agents may be added to sheath 14 for improved x-ray detectability. It is further envisioned that x-ray detectable element 10 may be of a heat laminated design.

FIGS. 3A-3C show x-ray detectable element 20 with radiopaque core 22 having an oval cross-section within sheath 24. FIG. 3B shows a longitudinal cross-sectional view of element 20 along plane b-b of FIG. 3A. FIG. 3C shows a longitudinal cross-sectional view of element 20 along plane c-c of FIG. 3A. The larger dimension of radiopaque core 22 in plane b-b is more visible under x-ray than the smaller dimension of core 22 in plane c-c. X-ray detectable element 20 having oval shaped radiopaque core 22 may be associated with an absorbent substrate in such a manner so as to produce a uniquely visible profile under x-ray.

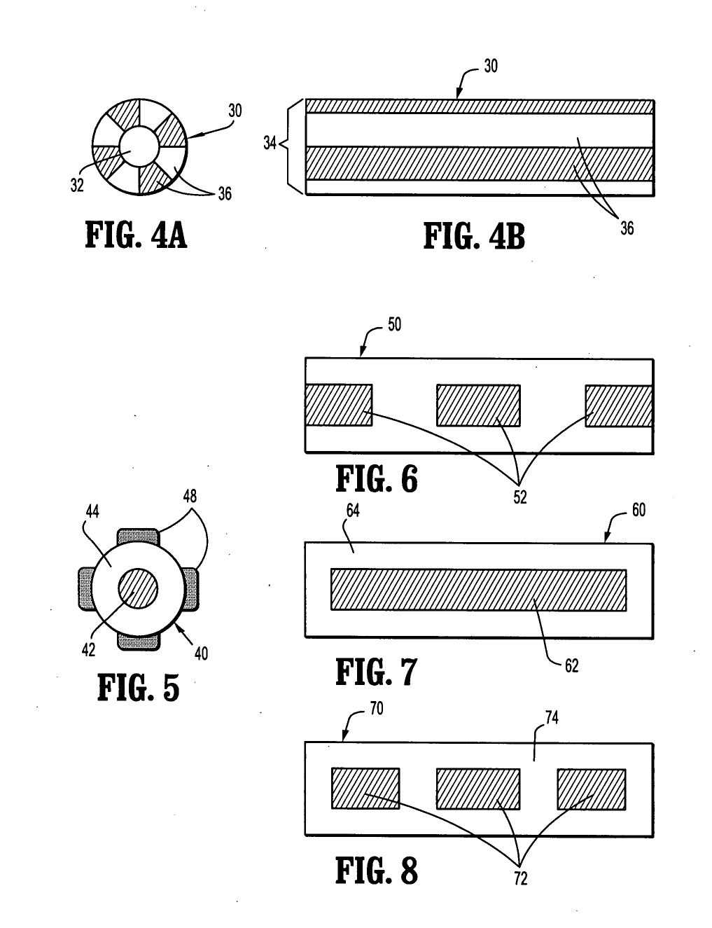

FIGS. 4A and 4B illustrate an alternate embodiment of the present disclosure. X-ray detectable element 30 includes radiopaque core 32 and sheath 34 co-axially mounted about the radiopaque core 32. Sheath 34 is preferably disposed about core 32 by extrusion, co-extrusion, or other methodologies familiar to those in the art. Elongated sheath 34 is portioned into radial segments or sheath regions 36 along the longitudinal axis. One or more sheath regions 36 may be of different colors and/or composition for increased visability and/or detectability. In one embodiment, elongated sheath 34 is radially partitioned into eight (8) sheath regions 36. Sheath regions 36 may be alternating in colors, such as blue or green or white, for facilitating in enhancing visibility and/or detecting the presences of blood, as discussed herein above. Sheath regions 36 composed of additional opacifying agent or agents increase the detectability of element 30 under x-ray, while the addition of magnetic material to any of sheath regions 36 would make element 30 to be detected by an electro-magnetic device. Sheath 34 may have any number of sheath regions 36 possessing any or all of the aforementioned properties.

FIG. 5 illustrates an alternate embodiment of the present disclosure. In accordance with this embodiment, x-ray detectable element 40 has radiopaque core 42, sheath 44, and an additional layer of material 48 partially encasing sheath 44. Additional layer 48 may be composed of any or all of the above mentioned materials or colors. Additional layer 48 may provide for increased visibility and/or detectability of element 40.

The radiopaque core may be of a continuous length within the sheath, as shown in FIGS. 1-4, or the radiopaque core may be intermittently spaced in the longitudinal direction, as depicted in FIG. 6. The intermittent radiopaque core 52 provides easier recognition of element 50 over other structures in the surgical field, such a pacemaker wires. In yet another embodiment, depicted in FIG. 7, x-ray detectable element 60 may have radiopaque core 62 fully encased within sheath 64, i.e., not extended to the ends of sheath 64. In a further embodiment, depicted in FIG. 8, x-ray detectable element 70 has intermittent radiopaque core 72 fully encased within sheath 74.

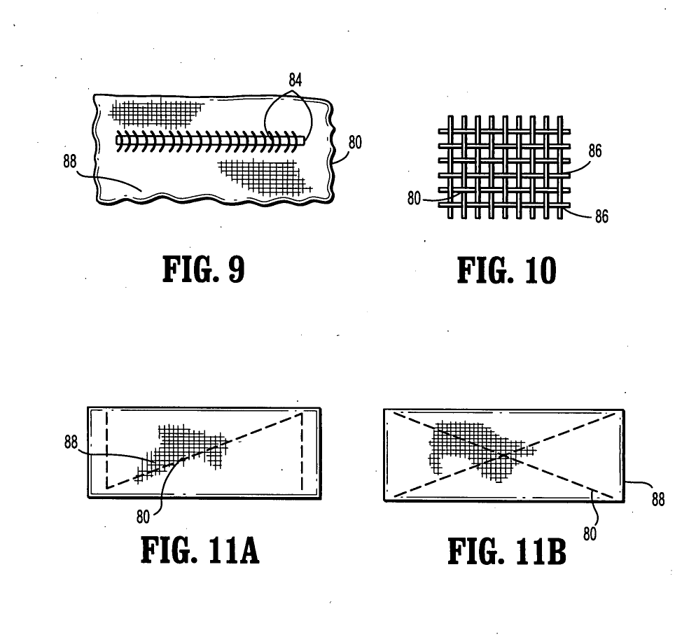

FIGS. 9-11 illustrate x-ray detectable element 80 in association with absorbent substrate 88. FIG. 9 depicts element 80 secured on absorbent substrate 88 through the use of stitching 84. FIG. 10 shows x-ray detectable element 80 woven amongst fibers 86 of absorbent substrate 88. Element 80 can be associated with absorbent substrate 88 by any of the aforementioned methods. Additionally, element 80 may be associated with substrate 88 through bonding and adhesion. FIGS. 11A and B show x-ray detectable element 80 woven into absorbent substrate 88 in various patterns for improved visibility and/or detectability. FIG. 11A depicts x-ray detectable element 80 woven into absorbent substrate 88 in the pattern of an extended “Z”. FIG. 11B depicts element 80 woven into absorbent substrate 88 in an “X” pattern. Absorbent substrate 88 may be a sponge, gauze, or the like and may be fabricated from, woven and non-woven, cotton, synthetic fibers, or the like.

It will be understood that various modifications may be made to the embodiments disclosed herein. Therefore, the above description should not be construed as limiting, but merely as exemplifications of preferred embodiments. Those skilled in the art will envision other modifications within the scope and spirit of the claims append hereto.

What we claim is:

1. An apparatus for absorbing fluids in body tissue, which comprises:
an absorbent substrate; and
a detectable element associated with the substrate, the element having an elongated radiopaque core and an extruded sheath at least partially encasing the core.

2. The apparatus of claim 1 wherein the absorbent substrate is a surgical sponge or gauze.

3. The apparatus of claim 1 wherein the radiopaque core comprises opacifying agent.

4. The apparatus of claim 3 wherein the opacifying agent is BaSO4.

5. The apparatus of claim 1 wherein the radiopaque core includes an elastomeric polymer.

6. The apparatus of claim 1 wherein the radiopaque core defines a general oval cross-section.

7. The apparatus of claim 1 wherein the radiopaque core is intermittently spaced along the longitudinal axis.

8. The apparatus of claim 1 wherein the radiopaque core is fully encased within the sheath.

9. The apparatus of claim 1 wherein the sheath is colored.

10. The apparatus of claim 1 wherein the sheath comprises an elastomeric material.

11. The apparatus of claim 1 wherein the sheath comprises a hydrophobic material

12. The apparatus of claim 1 wherein the sheath comprises one or more leachable antimicrobial agents.

13. The apparatus of claim 1 wherein the sheath comprises magnetic material.

14. The apparatus of claim 1 wherein the sheath includes a plurality of radial sheath regions.

15. The apparatus of claim 14 wherein one or more of the sheath regions are colored.

16. The apparatus of claim 14 wherein one or more of the sheath regions includes an opacifying agent.

17. The apparatus of claim 1 wherein the sheath is at least partially covered.

18. A method of making an x-ray detectable thread having a radiopaque core and at least a partially encasing sheath, the method comprising the step of:
extruding the radiopaque core within the sheath.

19. The method of claim 18 wherein 1 the step of extruding includes co-extruding the radiopaque core within the sheath.

20. An x-ray detectable element for use in association with an absorbent article, the element comprising:
an elongated radiopaque core; and
an extruded sheath at least partially encasing the core.
